(19) United States
(12) Patent Application Publication
(10) Pub. No.: US 2008/0090097 A1
(43) Pub. Date: Apr. 17, 2008

(54) CHEMICALLY AND PHYSICALLY TAILORED STRUCTURED THIN FILM ASSEMBLIES FOR CORROSION PREVENTION OR PROMOTION

(75) Inventors: Barbara Shaw, University Park, PA (US); Elzbieta Sikora, State College, PA (US); Mark Horn, State College, PA (US); Ryan C. Wolfe, Upper Saint Clair, PA (US)

Correspondence Address: MCKEE, VOORHEES & SEASE, P.L.C.;ATTN: PENNSYLVANIA STATE UNIVERSITY, 801 GRAND AVENUE, SUITE 3200, DES MOINES, IA, 50309-2721, US

(73) Assignee: THE PENN STATE RESEARCH FOUNDATION, University Park, PA (US)

(21) Appl. No.: 11/868,835

(22) Filed: Oct. 8, 2007

Related U.S. Application Data
(60) Provisional application No. 60/829,102, filed on Oct. 11, 2006.

(51) Int. Cl. B32B 15/00; A61F 2/06; A61L 33/00

(52) U.S. Cl. 428/686; 427/2.24; 623/1.38

(57) ABSTRACT

The composition of matter includes an alloy formed by vaporization of either magnesium or iron in continuation with one or more metals. This results in an array of alloy members each making up the alloy and extending from a base to an upper end. A fluid at least partially impregnates the spaces between the alloy members to the surrounding areas. A dissolving cap can either be included or deleted to prevent the fluid from diffusing until the cap is dissolved.

### CROSS-REFERENCE TO RELATED APPLICATIONS

This application claims priority to provisional application Ser. No. 60/829,102 filed Oct. 11, 2006, herein incorporated by reference in its entirety.

### BACKGROUND OF THE INVENTION

For corrosion prevention of metallic surfaces, surface treatments impart corrosion inhibiting and self-repairing capabilities. One way to achieve this involves the use of thin films that, through their designed physical and chemical properties, would deliver healing compounds to a damaged site. Thin films exhibiting such properties usually must be chemically and physically tailored over a range of length scales from micro to nano and could promote self-healing in a number of material systems.

Using different designs and chemistries, other thin films are particularly well suited for use and application in medical fields. For example, chemically and physically tailored thin films can be designed and produced to enhance certain specific characteristics that can either promote or deter cell attachment or determine the bioabsorbality of a medical device. Non-toxic, bioabsorbable biomaterials could be used for cardiovascular stents, orthopedic implants or as implantable carriers for local drug delivery systems (e.g., chemotherapeutic drugs at tumor sites). Specifically, advantages of bioabsorbable biomaterials as degradable or bioabsorbable cardiac biomaterials include: 1) elimination of restenosis caused by foreign body reactions to permanently implanted materials (this reduces the need for repeat procedures which are often needed with traditional stents), 2) avoiding risks associated with prolonged presence of a foreign material in the body (ultimately lowering health care costs), and 3) lowering risk of side effects. The bioadsorption of thin-film assemblies can be tailored by the thin-film itself or in conjunction with its drug-eluting capability.

Physically and chemically tailoring bioabsorbable biomaterials by forming channels or porosity on a metal surface in a uniform and inexpensive manner is a formidable challenge. Therefore, there is a need to develop a method and/or system for economically coating large surface areas with micro to nanometer-scale channels that could be used to deliver healing compounds. For instance, these materials can have their porosity and/or chemical composition graded as a function of thickness of the material. In addition, an open-cellular surface structure would permit easy transport of fluids and compounds and the structure of these openings could have their micro and nanostructure optimized to alter kinetics of drug release. Moreover, by depositing a metal vapor at varying incident angles, these channels can be formed directly, without using expensive lithographic techniques.

The benefits being derived from such self eluting of functional fluids from metal surfaces are innumerable. As previously noted, these materials might be used as biodegradable and bioabsorbable materials (in orthopedic, orthodontic and/or cardiovascular implants), as well as drug eluting biomaterials. Still, other applications could be envisioned where the materials store a fluidic compound configured to assist in healing damaged paint, masking thermal signatures and/or promoting electrical conductance. Still yet, these materials may act as a barrier or sacrificial layer/coating. Other considerations for such materials might include hydrogen storage (in fuel cells).

Thus, there is a need to develop an inexpensive method and/or system for manufacturing/producing continuously formed and/or capped thin film columns to contain surface healing compounds. In addition, there is a further need to develop non-toxic, bioabsorbable biomaterials for use in medical applications. Therefore, as there is a need for materials storing and eluting functional fluids and the production means exist to produce such materials within economies of scale, further disclosed is a method and system for the delivery of fluid through surfaces, including a specific application for tailoring metal surfaces to create bioabsorbable biomaterials for use in medical applications.

### BRIEF SUMMARY OF THE INVENTION

It is therefore a principle object, feature, or advantage of the present invention to provide an apparatus and method that solves problems in the art or improves over the state of the art.

It is a further object of the present invention to provide an alloy formed in combination with one or more metals from the group consisting essentially of Mg, Y, Ti, T, Nd, Zr, Zn, Al, Ce, Ca, and Cu.

It is a further object of the present invention to provide an alloy formed in combination with one or more metals from the group consisting essentially of Fe, Al, Cu, P, Cr, Ni, W, Ca, Mo, and N.

It is a further object, feature, or advantage of the present invention is to provide a process and method for manufacturing materials capable of fluid delivery.

It is still a further object, feature, or advantage of the present invention is to provide a process and method for manufacturing bioabsorbable biomaterials capable of use in medical applications.

It is yet another further object, feature, or advantage of the present invention to provide a bioabsorbable biomaterial constructed of an alloy magnesium or iron, presenting no toxicity to the human body.

It is another object, feature, or advantage of the present invention to provide a bioabsorbable biomaterial using magnesium, magnesium alloys, iron and/or iron alloys.

It is still another object, feature, or advantage of the present invention to provide a bioabsorbable biomaterial wherein the nano/micro structure and morphology are easily alterable to adjust dissolution rates and tailor with specific mechanical and physical properties.

It is yet another object, feature, or advantage of the present invention to provide a bioabsorbable biomaterial wherein the porosity and chemical composition are graded as a function of the thickness of the material.

It is a further object, feature, or advantage of the present invention to provide a bioabsorbable biomaterial having am open-cellular surface structure formed by spaces or columns between the magnesium or iron alloys to permit and promote transport of fluids and/or compounds.

It is still a further object, feature, or advantage of the present invention to provide a bioabsorbable biomaterial having a surface structure with micro and nano-structurally optimized openings for altering drug release kinetics.

It is yet another object, feature, or advantage of the present invention to provide a bioabsorbable biomaterial wherein the material is a rolled or coiled film, a ribbon, a coated wire, a micro-machined film or lithographed to form a pattern, a bulk vapor deposit with a structured surface, or a vapor deposited structured powder.

It is a further object, feature, or advantage of the present invention to provide a process wherein the material rollers are sufficiently sturdy to handle the desired thickness of steel.

Another object, feature or advantage of the present invention is to provide a material roll positioned off-center relative to the vapor source in order to form the nanocolumns on the material.

A further object, feature, or advantage of the present invention is to provide a material having nanocolumns formed when the vapor meets the substrate at an acute angle.

Another object, feature, or advantage of the present invention is to provide a means for forming a cap at the bottom of the roll where vapor impinges perpendicular to the substrate.

A still further object, feature, or advantage of the present invention is to provide a material wherein the ratio of column length to cap thickness is easily adjustable.

Yet another object, feature, or advantage of the present invention is to provide a material wherein if short columns and thicker caps are desired, the vapor source is simply moved towards the center of the roll.

Another object, feature, or advantage of the present invention is to provide a material wherein the application of the vapor is applied onto large, immovable substrates exceeding the weight or thickness if driven around the roller.

One or more of these and/or other objects, features, or advantages of the present invention will become apparent from the specification and claims that follow.

A composition of matter comprises an alloy formed by vaporization selected from one or more of the group consisting essentially of Mg, Y, Ti, T, Nd, Zr, Zn, Al, Ce, Ca, and Cu. An alternative form of the invention involves an alloy formed by vaporization selected from one or more of the group consisting essentially of Fe, Al, Cu, P, Cr, Ni, W, Ca, Mo, and N. An array of alloy members each makes up the alloy and extends from a base to an upper end. An upper body surface is provided by the combined upper ends of the alloy members. A plurality of channels are formed between the array of alloy members, the thicknesses of the channels being less than a micrometer. A fluid material is at least partially impregnating the channels and is capable of diffusing from the channels the surrounding area.

According to another feature of the present invention, the alloy is biodegradable within a human body.

According to another feature of the present invention, the alloy is deposited on the surface of the substrate.

According to another feature of the present invention, the alloy comprises a stent for insertion into a human.

According to another feature of the present invention, the fluid material is a medicine.

According to another feature of the present invention, the alloy is comprised of magnesium in the amount of 89% to 95%, yttrium in the amounts of 5%-9%, and less than 1% titanium.

According to another feature of the present invention, a capping layer is in covering relation over the upper body surface and prevents the fluid material from diffusing into the surrounding area.

According to another feature of the present invention, the capping layer is dissolvable in the human and dissolves after a predetermined period of time after which the fluid material is permitted to diffuse into the surrounding area.

According to another feature of the present invention, the alloy is within a human, the fluid material being a medication that at least partially prevents the capping layer from eluting outwardly of the alloy into the human. The capping layer after dissolving in the human permits the medicate to elute outwardly into the human.

A method is comprised of vaporizing an alloy comprising one or more of the metals selected from the group consisting essentially of Mg, Y, Ti, T, Nd, Zr, Zn, Al, Ce, Ca, and Cu. An alternative form of the invention involves vaporizing an alloy comprising one or more metals selected from the group consisting essentially of Fe, Al, Cu, P, Cr, Ni, W, Ca, Mo, and N. The vaporization is continued until an alloy is formed comprising an array comprised of a plurality of alloy members each extending from a base to an upper body end. A plurality of spaces are provided between the alloy members. The spaces are impregnated with a fluid material for the purpose of diffusing from the spaces to the surrounding area.

### BRIEF DESCRIPTION OF THE DRAWINGS

FIG. 1 is a schematic electronic image diagram of a sectional view of the present invention.

FIG. 2 is a schematic diagram of one proposed method of making the invention of FIG. 1.

FIG. 3 is an enlarged perspective electronic image view of a section of the one modified form of the present invention.

FIG. 4 is one enlarged electronic image of a sectional view of a second modified form of the present invention.

FIG. 5 is an enlarged electronic image of a sectional view of the third modified form of the present invention.

FIG. 6 is an enlarged perspective electronic image view of a section of a fourth modified form of the present invention.

FIG. 7 is a schematic illustration of a fifth modified form of the present invention.

FIG. 8 is an enlarged sectional view of a wire modified form of the present invention.

FIG. 9 is an enlarged plan view of a modified form of the present invention.

FIG. 10 is an enlarged perspective view of a stent made by the present invention.

FIG. 11 is an enlarged perspective view of a solid tube made by the present invention.

### DETAILED DESCRIPTION OF THE PREFERRED EMBODIMENT

The present invention relates to an apparatus and method for storing and delivering functional fluids to and through a surface and having specific application for use as a bioabsorbable biomaterial in varying medical applications.

The material consists of an array of metal columns, which are formed on a substrate that is angled with respect to metal vapor source. The voids between columns could be used to store and transmit surface healing compounds.

In order to store the surface healing compounds, the intercolumnar void network must be sealed. Therefore a capping layer can be made of the same material as the columns, resulting in the sealed structure. Since the spaces between the columns are interconnected, the self-healing fluid will flow across the two-dimensional area of the surface, as opposed to the one dimensional flow of biomimetic systems which simulate the flow of blood through a vessel.

In the past, production costs for previous methods of producing STFs (Sculptured Thin Films) have been inhibitive. In order for these coatings to be practical, they must be produced in an inexpensive manner. The material could be coated by continuous feeding around a roll above one metal vapor source. Such continuous feeding techniques are common place in manufacturing and necessarily efficient. These processes are well established, evidenced by the enormous amount of materials currently processed in this manner, including such items as potato chip bags and shiny gift wrapping.

Coatings can be formed by physical vapor deposition that have 10-90% interconnected porosity. These Sculptured Thin Films (STFs) have been developed mainly for their unique optical properties, but have potential for application in the corrosion field as well.

Referring to FIG. 1, a thin film assembly 10 includes a capping layer 12, a plurality of nanocolumns 14, a self healing fluid 16, and a substrate 18. An initial alloy member or base 17 is provided at the lower end of nanocolumns 14. The substrate 18 may be removed after it is initially utilized to create the vaporization layer of 12, 14, 16. The capping layer 12 is in covering relation over the nanocolumns 14 and over the self healing fluid 16. The self healing fluid 16 is impregnated in a plurality of channels (not shown) and is capable of diffusing or eluting outwardly to the surrounding area between the nanocolumns 14. The direction of dissolving or eluting is designated by the numeral 19.

The nanocolumns 14 are made by the combination of several alloys deposited by vaporization at a plurality of angles to the substrate 18. These alloys are comprised of the following: one of the alloys is formed by one or more of the metals selected from the group consisting essentially of Mg, Y, Ti, T, Nd, Zr, Zn, Al, Ce, Ca, and Cu. Similarly, the alloy may be comprised of one or more of the metals selected from the group consisting essentially of Fe, Al, Cu, P, Cr, Ni, W, Ca, Mo, and N.

The capping layer 12 is dissolvable in the human body, and the self healing fluid 16 can be one or more medications. The capping layer 12, while dissolvable in the human body, prevents the medications 16 from diffusing in the direction of the arrow indicated by 19. However, after the dissolution of the capping layer 12, the medication 16 is permitted to move outwardly in the arrow indicated by 19.

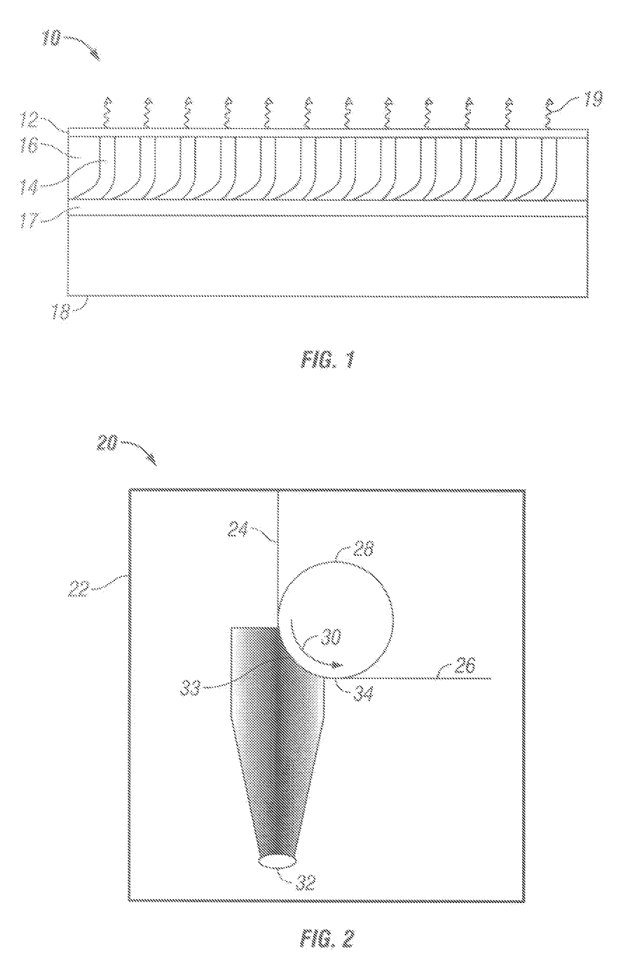

FIG. 2 shows an example of a method for applying the layered material to a substrate. This is only one of several that may be used to vaporize one or more metals and deposit them on a substrate. The system is referred to as a deposit system 20. While various types of deposit systems may be utilized, the present system 20 is typical. It includes an enclosed area 22 which has within it an uncoated sheet 24 which is later coated as indicated at 26. A roller 28 is provided around which the sheets 24, 26 extend. Roller 28 is rotatably mounted for rotation about an arrow indicated by 30. A vapor source 32 is provided and extends upwardly to a vaporization point which is approximately at the area indicated by the numeral 33. The capping operation 34 is provided on the lower end of roller 28. Capping operation 34 may be included or may be deleted as desired.

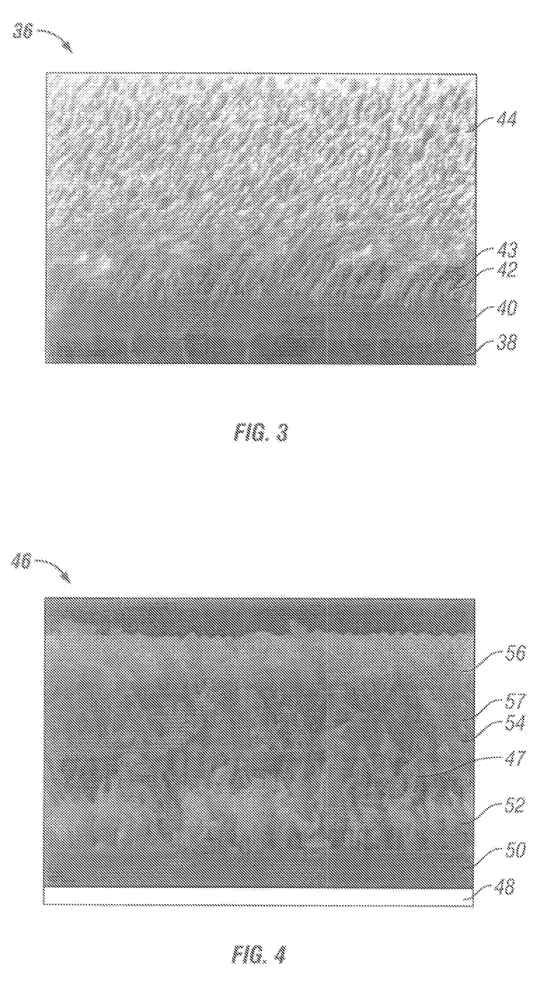

A modified form of the film deposit 36 is shown in the perspective view of FIG. 3. This is an STF which includes a substrate 38 and a metal base 40. Extending upwardly from the metal base 40 are a plurality of nanocolumns 42. The length of the vapor source 32 to the roller 28 as well as the length of time and the angle at which the vapor source 32 is provided, creates the various types of nanocolumns 42. The upper ends of the nanocolumns 42 are combined to form a surface area 44. A plurality of channels 43 are provided in the spaces between the nanocolumns 42. The nanocolumns 42 are at least less than a micron in diameter, but preferably less than a nanocolumn in diameter. As with prior versions of the invention, the substrate 38 may be removed after it has been deposited on by the alloy 40 and nanocolumns 42.

Referring to FIG. 4, a sectional view is provided for a modified form of the film deposit 46. Form 46 includes a substrate 48 and a metal base 50 from which extend a plurality of nanocolumns 47. The nanocolumns begin at their lower ends with a more porous base 52, and then extend into a more dense layer 54, and finally into a top layer 56 which is more porous. The modified form 46 is provided with a plurality of channels 57 that are vacant. There is no cap on the layer 46.

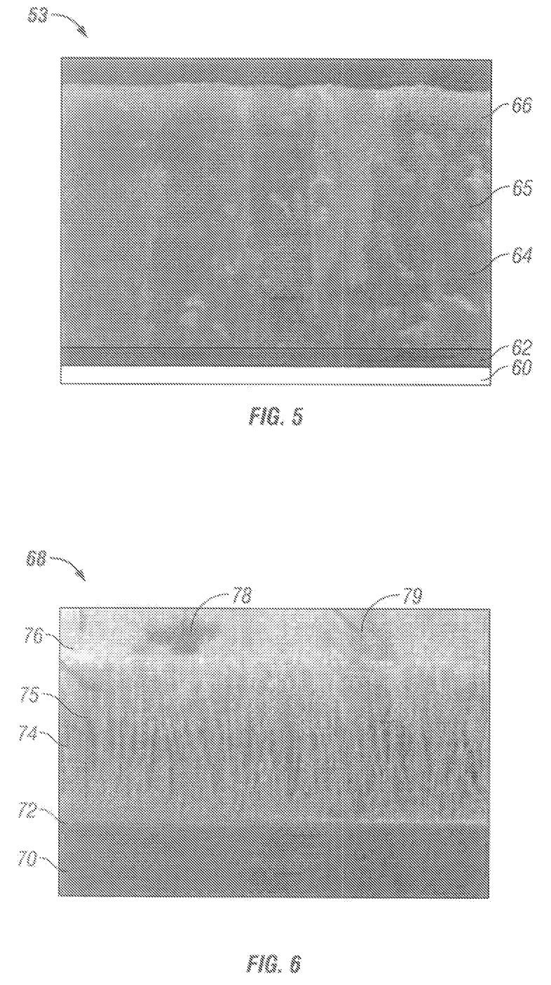

Referring to FIG. 5, a modified form of the film deposit 58 is provided which includes a substrate 60 and an alloy 62. Nanocolumns 64 are provided and channels 65 are vacant between the upstanding layers or nanocolumns 64. A cap 66 is provided at the upper end of the nanocolumns 64. The cap is dissolvable in the human body, and once dissolved, permits the medications in the channels 65 to diffuse into the surrounding human body.

A modified form of the film deposit is shown in FIG. 6 and is designated by the numeral 68. Thin film deposit 68 includes a substrate 70 and an alloy layer 72. A nanostructure 74 extends upwardly from the metal layer 72 and a plurality of channels 75 are provided that are vacant. The channels may be provided with a medication that can diffuse outwardly from the channels 75. A surface area 76 is provided by the upper ends of the nanostructure members 74 and includes voids 78, 79 therein. The voids and the channels between the nanostructure 74 provide a means for medication to be impregnated in the material. The device 68 and FIG. 6 are shown without a cap, but a cap may be provided.

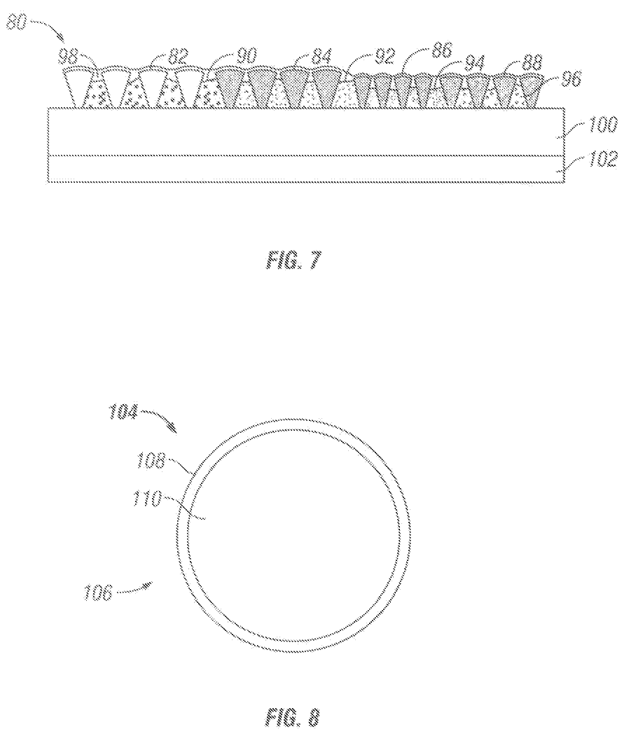

Referring to FIG. 7, a schematic system 80 is shown. The schematic system 80 shows first alloy at 82, second alloy at 84, third alloy at 86, and a fourth alloy at 88. A first drug 90 is provided between the alloys 82, a second drug 92 is provided between the second alloy 84, and a third drug designated by the numeral 94 is shown between the third alloy at 86. Finally a fourth drug is designated by the numeral 96 and extends between the fourth alloys 88. By providing different alloys with varying dissolution rates, the system of FIG. 7 is capable of delivering fluids to the surface over differing periods of time. Note that the width and length of the nanocolumns can be varied. This figure illustrates the topographical structures that could be used to create a multiple drug solution or dissolutions system. The spaces between the alloys 82, 84, 86, and 88, may be provided with different types of drugs or medications as indicated by the numerals 90, 92, 94, and 96. A capping layer 98 is provided over the entire surface of the nanostructures, but it may be eliminated as desired. An alloy base 100 which forms the base for alloys 82, 84, 86, 88 is mounted on a substrate 102.

All of the above structures may comprise magnesium only or an alloy of magnesium in combination with one or more metals. They also may comprise iron only or one or more metals alloyed with iron utilized as indicated above.

FIG. 8 illustrates a wire coating 104 which is shown in section. Wire coating 104 includes an arrow 106 showing the vapor direction. The wire 104 is rotated to create the deposit as indicated above. An alloy coating 108 is provided on the surface of wire 110.

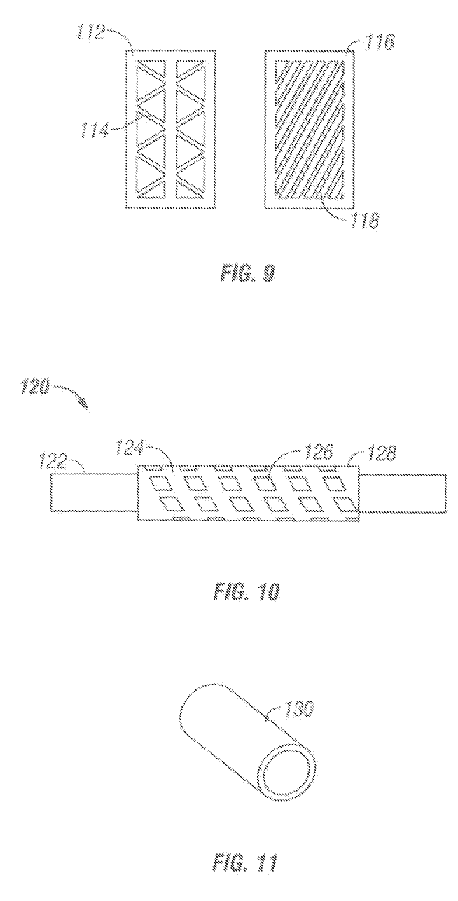

Referring to FIG. 9, a flat sheet 112 includes a plurality of open areas 114. Similarly, a flat sheet 116 is shown to include a plurality of open areas 118. The flat sheets 112, 116 may be formed into a typical stent.

FIG. 10 shows a modified stent 120 formed about a glass rod 122. The member 120 is provided with a photo resist 124 which prevents the metal alloy from being etched. However, the etched area at 126 is permitted to become etched away and results in a stent 128. Stent 128 may be provided with a medication and a cap such as described previously, or the cap may be eliminated. Thus, the stent permits the eluting of the medication from its impregnated source between the nanostructures outwardly to the area surrounding the stent 128.

Referring to FIG. 11, a modified form 130 is shown which utilizes a tube that is nonpatterned.

The material consists of an array of metal columns, which are formed on a substrate that is angled with respect to metal vapor source. The voids between columns could be used to store and transmit surface healing compounds.

Depending upon the alloys selected, the vapor deposited nonequilibrium magnesium or iron alloys exhibit a wide range of corrosion rates and can be deposited in sculptured form. Two potential applications for sculptured vapor deposited magnesium or iron alloys include 1) biodegradable and bioabsorbable materials (in orthopedic, orthodontic and/or cardiovascular implants), and 2) materials for hydrogen storage (in fuel cells). Because magnesium or iron and its alloys exhibit a high strength-to-weight ratio they are well suited for use in automotive, aerospace and electronic applications as advanced light-weight materials. Magnesium, iron, magnesium alloys and iron alloys are also used as battery electrodes, sacrificial anodes, and hydrogen storage materials, as well. For example, as a biomaterial, magnesium or iron is well suited and facilitates the biodegradability of implants made of magnesium alloys because of its high corrosion rate.

Accordingly, new techniques like vapor deposition are welcomed and broadly embraced because it allows the production of magnesium alloys with tailored mechanical properties and lower corrosion rates. For example, a magnesium alloy may be used as the substrate shown in FIG. 5. This is particularly important in biomaterials applications where uniform corrosion is desirable. Sculptured vapor deposits of Mg alloy substrates seem to be particularly good candidates for new biomaterials. Using vapor deposition, the chemical composition of the alloys can be easily manipulated (since vapor deposition allows nonequilibrium alloys). Moreover, nano/micro structures and morphologies of the substrate may be altered for various applications, including medical applications. For instance, the magnesium substrate could have graded porosity using vapor deposition, making it even more osteoconductive. In medical applications, the open-cellular structure would permit the growths of new bone and/or transport of fluids and compounds. Also, the strength and the modulus could be adjusted (through varying degree of porosity) to match the strength of the natural bone. For example, application of structured vapor deposited Mg as biodegradable stents would permit to production of sculptured surfaces with nanotopography and could positively influence biointegration simple because the stents (or bone implants) could serve as a local drug delivery system. In particular, the standing voids between the nanocolumns, as shown in FIG. 5, could store and transmit different compounds. One example of this could include compounds facilitating the incorporation of implants into host's body, like growth factors or compounds preventing hostile reactions (like antibiotics). In any case, the self-healing fluid incorporated into the nanocolumns could be encapsulated within the columns using a capping layer, as illustrated in FIG. 5.

Hydrogen storage materials are another potential application of vapor deposition forming nanocolumns on a substrate, as shown in FIGS. 3-5 and 8-11. Using vapor deposition, magnesium thin films could be formed and simultaneous control of their chemical composition and microstructure can be achieved. The ability to form nanostructured films by physical vapor deposition is especially relevant since it has been established that nanometric structures enhance hydriding. Not only would the surface area be larger for a sculptured thin-film, but the diffusion paths for hydrogen would be shorter; therefore, eliminating a major concern encountered when pure magnesium is used as the substrate material, shown in FIGS. 3-5 and 8-11. In particular, the voids between columns of sculptured vapor deposits (as illustrated in FIGS. 3-6 and 8-11) could provide more space for storing hydrogen, and serve as channels for the transportation of hydrogen from the surface into the bulk and vice versa. In addition to the use of magnesium as the substrate, the physical vapor deposition system could be used to create new, nanocrystalline thin film alloys containing magnesium, nickel and aluminum or lithium. This is especially relevant since vapor deposition allows for the creation of nonequilibrium alloys having a variety of compositions. Also, vapor deposited films could be hydrogenated by ion beam assisted deposition. In this approach, hydrogen is incorporated into the nanocolumn structure of the alloy during deposition.

Ultimately, in order to store any of the suggested self-healing fluids or compounds, the intercolumnar void network must be sealed. This capping layer can be made of the same material as the columns, resulting in the structure shown in FIG. 5. However, any of the structures 3-11 may be utilized with a cap. Since the spaces between the columns are interconnected, the self-healing fluid will flow across the two-dimensional area of the surface, as opposed to the one dimensional flow of biomimetic systems which simulate the flow of blood through a vessel.

In order for these coatings to be practical, they must be produced in an inexpensive manner. One method for cheaply producing or manufacturing continuously form capped metal columns for containing surface healing compounds is illustrated in FIG. 2. For example, one embodiment of production or manufacturing would be to continuously feed the material to be coated around a roller or belt above of metal vapor source, as illustrated in FIG. 2. This is a well established process, evidenced by many commercial and industrial applications, such as the enormous amounts of material currently processed in this manner to be used for items such as potato chip bags and shiny gift wrapping. The only modifications that need to be made to the process are that the rollers must be sturdy enough to handle the desired thickness of steel or substrate material. Such systems are currently used in the processing and manufacturing of sheet metal. Thus, the roller in FIG. 2 could be of similar specification and design as those used in the sheet metal processing and manufacturing industries. A motor (not shown) could drive the roller at a rate amenable to vapor deposition. The vapor source would be similar to those that are commercially available and known in the industry for applying vapor by deposition to a substrate. The vapor source would be positioned at an oblique angle and off-center with respect to the roller in order to form the nanocolumns. The nanocolumns are formed on the left portion of the roll in FIG. 2 everywhere the vapor meets the substrate at an acute angle. Nearer the bottom of the roller, the vapor impinges the substrate at angles perpendicular to the substrate; thus, a cap is formed over the top of the nanocolumns.

The ratio of column length to cap thickness is adjustable. If a thicker cap is desired relative to the nanocolumns, this is accomplished by increasing the proportion of the vapor flux impinging on the substrate at an angle perpendicular to the roller over the proportion of the vapor flux impinging on the substrate at an acute angle; in other words, moving the vapor source closer toward a line extending perpendicularly downward from the center of the roller. Conversely, if taller nanocolumns are desired relative to the capping layer (a thinner capping layer), this is accomplished by increasing the proportion of the vapor flux impinging on the substrate at an acute angle over the proportion of the vapor flux impinging on the substrate at perpendicular angles; in other words, moving the vapor source away from, to the left of a line extending perpendicularly downward from the center of the roller. For example, in the case where short columns and thicker caps are desired, the vapor source can simply be moved towards the center of the roller thereby increasing the proportion of the vapor flux impinging on the substrate at perpendicular angles. Even yet, modifications to this particular embodiment may allow for the coating or vapor flux to be applied onto large, immovable substrates that are too large to be trained over a roller. This could be accomplished by making the vapor source mobile with respect to the surface being coated. The angle of the vapor source relative to the substrate being coated could be changed to realize taller or shorter nanocolumns and/or a thicker or thinner capping layer.

In its completed form, this system will allow for the delivery of self-healing fluids to a damaged site in a coating. In addition to the self-healing capabilities, the coating can also act as a barrier or sacrificial coating, depending upon the coating material and the substrate being used. A method has been proposed to form these coatings in an economical manner using a well-developed technology.

In one specific embodiment of the present application, magnesium and iron are particularly well suitable as bioabsorbable biomaterials since they are non-toxic and they are beneficial to human body. Sculptured vapor deposited metals and alloys (such as magnesium, magnesium alloys, iron, and iron alloys) are good candidates for new bioabsorbable biomaterials (such as stents) because the chemical composition of the alloys and their nano/micro structures and morphologies could be easily altered specifically to adjust dissolution rates and tailor mechanical and physical properties. For instance, these materials can have their porosity and/or chemical composition graded as a function of thickness of the material. In addition, an open-cellular surface structure would permit easy transport of fluids and compounds and the structure of these openings could have the micro and nanostructure optimized to alter kinetics of drug release.

The structured vapor deposited material that forms the biomaterial could take on any one of several forms including (but not limited to): a rolled or coiled film, a ribbon, a coated wire, a film that is micro-machined or lithographed to form a pattern, a bulk vapor deposit with a structured surface, or vapor deposited structured powders. A photograph of one example of a structured deposit on a wound wire is shown in FIG. 4. The wire is treated or tailored using the same process for treating surfaces, previously disclosed above. FIG. 2 shows SEM images of the vapor deposited surface and illustrates changes in the morphology of the deposit with position on the circumference of the wire. Moreover, this change in morphology as a function of position on the wire is controlled by altering the position of the vapor source relative to the wire, as previously discussed above.

The preferred embodiment of the present invention has been set forth in the drawings and specification, and although specific terms are employed, these are used in a generic or descriptive sense only and are not used for purposes of limitation. Changes in the form and proportion of parts as well as in the substitution of equivalents are contemplated as circumstances may suggest or render expedient without departing from the spirit and scope of the invention as further defined in the following claims.

What is claimed is:

1. A composition of matter comprising:
an alloy being formed by vaporization with one or more of the metals selected from the group consisting essentially of Mg, Y, Ti, T, Nd, Zr, Zn, Al, Ce, Ca, and Cu;
an array of alloy members each making up the alloy and extending from a base to an upper end;
an upper body surface formed by the combined upper ends of the alloy members;
a plurality of channels formed between the array of alloy members, the thicknesses of the channels being less than a micrometer;
a fluid material at least partially impregnating the channels and being capable of diffusing from the channels to the surrounding area.

2. The composition of matter according to claim 1 and further characterized by the alloy being biodegradable within a human body.

3. The composition of matter according to claim 1 and further characterized by a substrate, the alloy being deposited on the surface of the substrate.

4. The composition of matter according to claim 1 the alloy comprises a stent for insertion to a human.

5. The composition of matter according to claim 1 wherein the fluid material is a medicine.

6. The composition of matter according to claim 1 wherein the alloy is comprised of magnesium in the amount of 89% to 95%, yttrium in the amounts of 5-9%, and less than 1% titanium.

7. The composition of matter according to claim 1 wherein a capping layer is in covering relation over the upper body surface and prevents the fluid material from diffusing into the surrounding area.

8. The composition of matter according to claim 7 wherein the capping layer is dissolvable in the human and dissolves after a predetermined period of time after which the fluid material is permitted to diffuse into the surrounding area.

9. The composition of matter according to claim 1 wherein the alloy is within a human, the fluid material being a medication being at least partially being prevented by the capping layer from eluting outwardly of the alloy into the human, the capping layer after dissolving in the human permitting the medication elute outwardly into the human.

10. The composition of matter according to claim 1 wherein the alloy includes hydrogen therein.

11. A composition of matter comprising:
an alloy being formed by vaporization formed with one or more of the metals selected from the group consisting essentially of Fe, Al, Cu, P, Cr, Ni, W, Ca, Mo, and N;
an array of alloy members each making up the alloy and extending from a base to an upper body surface;
a plurality of channels formed between the array of alloy members, the thicknesses of the channels being less than a micrometer and the upper body ends together comprising an upper body surface;
a fluid material at least partially impregnating the channels for the purpose of diffusing from the channels to the surrounding area.

12. The composition of matter according to claim 11 wherein the alloy includes hydrogen therein.

13. A method of making a film comprising:
vaporizing an alloy comprising one or more of the metals selected from the group consisting essentially of Mg, Y, Ti, T, Nd, Zr, Zn, Al, Ce, Ca, and Cu;
continuing the vaporization until an alloy is formed comprising an array comprised of a plurality of alloy members each extending from a base to an upper body end, a plurality of spaces being between the alloy members;
impregnating the spaces between the alloy members with a fluid material for the purpose of diffusing from the spaces to the surrounding area.

14. The method according to claim 13 comprising biodegrading the alloy within a human body after installation of the alloy within the human body.

15. The method according to claim 13 and wherein the vaporization step comprises depositing the alloy on a substrate.

16. A method for making a film comprising:
vaporizing an alloy comprising one or more of the metals selected from the group consisting essentially of Fe, Al, Cu, P, Cr, Ni, W, Ca, Mo, and N;
continuing the vaporization until an alloy is formed comprising an array comprised of a plurality of alloy members each extending from a base to an upper body end, a plurality of spaces being between the alloy members;
impregnating the spaces between the alloy members with a fluid material for the purpose of diffusing from the spaces to the surrounding area.
